(19) United States
(12) Patent Application Publication
(10) Pub. No.: US 2020/0286606 A1
(43) Pub. Date: Sep. 10, 2020

(54) METHOD FOR ENHANCING PATIENT COMPLIANCE WITH A MEDICAL THERAPY PLAN AND MOBILE DEVICE THEREFOR

(71) Applicant: SIPPA Solutions, LLC, Bayside, NY (US)

(72) Inventor: Bon K. Sy, Bayside, NY (US)

(21) Appl. No.: 16/651,418

(22) PCT Filed: Oct. 1, 2018
(86) PCT No.: PCT/US18/53748

§ 371 Date: Mar. 27, 2020

Related U.S. Application Data
(60) Provisional application No. 62/565,812, filed on Sep. 29, 2017.

(51) Int. Cl. G16H 20/00; G16H 10/60; G16H 10/20; G16H 50/70; G16H 40/67; G16H 80/00; G06Q 30/02; G16H 50/20; G06F 21/60; G06F 21/62; H04L 9/32; H04N 7/14

(52) U.S. Cl. CPC G16H 20/00; G16H 10/60; G16H 10/20; G16H 50/70; G16H 40/67; H04N 7/147; G06Q 30/0201; G16H 50/20; G06F 21/602; G06F 21/6245; H04L 9/3231; G16H 80/00

(57) ABSTRACT

A method for enhancing patient compliance with a medical therapy plan. The method compiles the information from multiple medical records into a single consolidated medical record, and stores it on a single mobile device. Medical devices supply supplemental data directly to the mobile device. Surveys, that help a patient to identify behavioral indicators, are distributed before and after supplemental data is accumulated. Comparison of the surveys quantifies changes in key disposition values of the patient. A customized message is displayed that addresses changes in dispositions that are key to compliance with the medical therapy plan.

### CROSS-REFERENCE TO RELATED APPLICATIONS

This application claims priority to and is a non-provisional of U.S. Patent Application 62/565,812 (filed Sep. 29, 2017), the entirety of which is incorporated herein by reference.

### STATEMENT OF FEDERALLY SPONSORED RESEARCH OR DEVELOPMENT

This invention was made with Government support under grant number 1648780 awarded by the National Science Foundation. The government has certain rights in the invention.

### BACKGROUND OF THE INVENTION

The total cost of health care services reported by the Center for Disease Control (CDC) in 2012 was $2.7 trillion. Of these expenditures, 86% were attributed to patients with chronic disease. Approximately 50 percent of the US population has one or more chronic disease. Chronic disease is the single largest burden to the health care system, accounting for 81% of hospital admissions, 91% of all prescriptions and 76% of physician visits. In a recent CDC National Diabetes Statistics Report, 30.2 million people in the United States are afflicted with chronic diabetes. Most of these people suffer from either obesity, high blood pressure, high cholesterol, physical inactivity, smoking or a combination of these conditions. The direct and indirect cost of diabetes on the health care system amounted to $245 billion, with each patient costing the system $13,700 per year, which is 2.3 times the average of all patients.

Research has shown time and again that patient engagement leads to better care outcome and reduces cost burden on the healthcare system. However, patient engagement relies on the readiness, and willingness, to take ownership on self-health management. Yet there is a lack of quantitative models to assist on understanding the alignment between the delivery of digital health service and motivation indicators to engage an individual in self-management of chronic diseases.

Telehealth has been rapidly growing in the last few years and is projected to reach a market size of $30B. Remote patient monitoring (RPM) falls under the umbrella of telehealth and aims to reduce the risk of Emergency Room (ER) visits and to slow down the progression of a chronic disease through self-monitoring while the data gathered by patients at home are made instantly available for the care providers at a remote location. A RPM program typically requires a patient to be assessed prior to an enrollment. The assessment is to determine whether a patient is ready to be activated for self-monitoring. Various assessment tools are currently available.

Stanford has published a set of evaluation tools for diabetic self-management. The evaluation tools have survey questions, scales, and the statistics on the score, such as average and standard deviation, from the population of their study. PAM13 is a commercial assessment tool that could be licensed from Insignia Health. PAM13 is a 13-question survey for patient activation measure. PAM13 and Stanford assessment tools both place a focus on self-efficacy measure. The readiness of a patient for an activation in self-management is linked to self-efficacy. Other theory-based behavior models are known-Natural Helper Model, Diffusion of Innovations Model, Theories of Organizational Change, Community Coalition Action Theory, Social Marketing Model, Precede-Proceed Model, Motivational Interviewing, Stages of Change Model, Social Learning Interpersonal Theory, Consumer Information Processing Model, Implementation Individual Intentions Models, and Health Belief Model. While these models were discussed in terms of the theories behind, applications, and limitations for disease management, these models are not necessarily focused on individual level. For example, Theory of Organizational Change Model targets disease management programs in the community level and focuses on the planning and implementation of population-based interventions that influence social norms and structures.

Recently, Theory of Planned Behavior Model, Trans-theoretical Model of Behavior Change, Health Belief Model, and IMB (Information Motivation and Behavior Skill) Model have been applied to specific intervention of chronic diseases, and have shown clinical efficacy. It was suggested that individuals perceiving risk of a condition are more likely to engage in behavior change to reduce risk. Thus perceived health risk, resulting in change of attitude and behavior are proponents for stronger intentions to be physically active and to maintain a healthy diet.

The discussion above is merely provided for general background information and is not intended to be used as an aid in determining the scope of the claimed subject matter.

### BRIEF DESCRIPTION OF THE INVENTION

A method for enhancing patient compliance with a medical therapy plan. The method compiles the information from multiple medical records into a single consolidated medical record, and stores it on a single mobile device. Medical devices supply supplemental data directly to the mobile device. Surveys, that help a patient to identify behavioral indicators, are distributed before and after the supplemental data is accumulated. Comparison of the surveys quantifies changes in key disposition values of the patient. A customized message is displayed that addresses changes in dispositions that are key to compliance with the medical therapy plan.

In a first embodiment, a mobile computing device configured to execute a method for enhancing patient compliance with a medical therapy plan is provided. The method comprising steps of: compiling a personal medical record that is stored on the mobile computing device owned by the patient, the personal medical record comprising at least one of medical data, health-wellness data or dietary data of the patient; constructing a first survey comprising a first plurality of questions; receiving a first set of answers concerning the first survey; wirelessly connecting the mobile computing device to at least one digital medical device; accumulating medical data over a period of at least four days from the at least one medical device and updating the personal medical record with the medical data, the health-wellness data or the dietary data; constructing a second survey comprising a second plurality of questions; receiving a second set of answers from the patient concerning the second survey, wherein the second survey is spaced at least four days after the first survey; quantifying a change in a disposition of the patient by comparing the first set of answers to the second set of answers; displaying a message to the patient on the mobile computing device wherein the message is customized based on the second set of answers and the change in disposition.

In a second embodiment, a mobile computing device configured to execute a method for enhancing patient compliance with a medical therapy plan is provided. The method comprising steps of: compiling a personal medical record that is stored on the mobile computing device owned by the patient, the personal medical record comprising at least one of medical data, health-wellness data or dietary data of the patient; constructing a first survey comprising a first plurality of questions; assigning each question in the first plurality of questions a first motivation weight, a first intention weight, a first attitude weight and a first ownership weight; receiving a first set of answers concerning the first survey; quantifying (1) a baseline motivation score using the first set of answers and the first motivation weight (2) a baseline intention score using the first set of answers and the first intention weight (3) a baseline attitude score using the first set of answers and the first attitude weight (4) a baseline ownership score using the first set of answers and the first ownership weight; wirelessly connecting the mobile computing device to at least one digital medical device; accumulating medical data over a period of at least four days from the at least one medical device and updating the personal medical record with the medical data; constructing a second survey comprising a second plurality of questions; assigning each question in the second plurality of questions a second motivation weight, a second intention weight, a second attitude weight and a second ownership weight; receiving a second set of answers from the patient concerning the second survey, wherein the second survey is spaced at least four days after the first survey; quantifying (1) an updated motivation score using the second set of answers and the second motivation weight (2) an updated intention score using the second set of answers and the second intention weight (3) an updated attitude score using the second set of answers and the second attitude weight (4) an updated ownership score using the second set of answers and the second ownership weight; quantifying a change in the motivation, a change in the intention, a change in the attitude and a change in the ownership based on a comparison of (1) the baseline motivation score and the updated motivation score, (2) the baseline intention score and the updated intention score, (3) the baseline attitude score and the updated attitude score, and (4) the baseline ownership score and the updated ownership score, respectively; displaying a message to the patient on the mobile computing device wherein the message is customized based on the second set of answers and the change in the motivation, the change in the intention, the change in the attitude and the change in the ownership.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a flow diagram depicting one method for enhancing patient compliance with a medical therapy plan;

FIG. 2 depicts an architecture of a model for processing survey answers for use with the method; and

FIG. 3 illustrates a model showing an alignment between the motivation indicator of the individuals with chronic conditions and a customized message provided by a reminder service.

### DETAILED DESCRIPTION OF THE INVENTION

In order to gauge how effectively a patient could be engaged in self-health management including self-monitoring, an assessment tool for RPM ideally should determine (1) the level of readiness in terms of motivation and skill, (2) the likelihood of behavior change overtime, and (3) the underlying relationship linking motivation, attitude and intention to behavior change.

This disclosure provides a quantitative model grounded on a behavior theory that has already been applied and shown efficacy in clinical studies. More specifically, such a quantitative model should help to reveal the linkage among behavior constructs, and should provide inference power to gain insights into not just the level of readiness in terms of motivation and skill for self-management, but the underlying relationship linking motivation, attitude, and intention to behavior change affected by the digital health services delivered in a mobile platform. Towards this end, this disclosure uses the Theory of Planned Behavior as a starting point for the development of a quantitative model just mentioned.

The Theory of Planned Behavior (TPB) provides a model to manifest the relationship among attitude, subjective norm, perceived behavioral control, intention and behavior. TPB is modeled through expectancy-value and assumes the best single predictor of an individual's behavior is an intention to perform that behavior. The intention in turn depends on the attitude of an individual (positive or negative evaluation of performing a behavior); the subjective norm (perception of whether relevant others think one should or should not perform the behavior); and perceived behavioral control (perception of the ease or difficulty of carrying out a behavior).

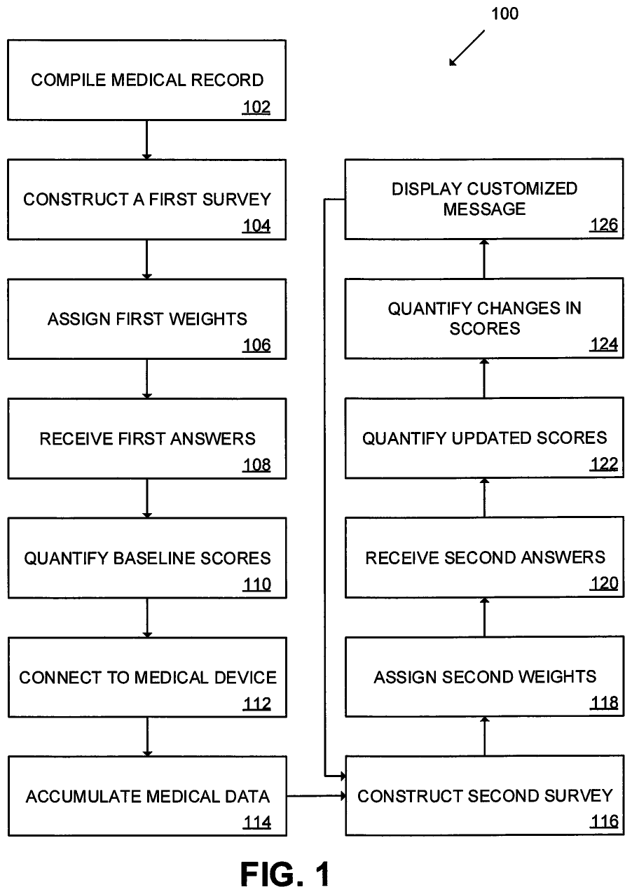

Referring to FIG. 1, a method 100 is showing for enhancing patient compliance with a medical therapy plan. Method 100 utilizes a mobile computing device to execute method 100. Examples of suitable mobile computing devices include smartphones, tablets and other similar devices. Mobile computing devices have the capability to place telephone calls, utilize video chat and store/update data files. The medical therapy plan is a treatment plan for a medical condition, such as a chronic medical condition. Examples of medical therapy plan include monitoring certain vital signs on a certain schedule and/or engaging in exercise on a certain schedule.

In step 102 of method 100, a personal medical record is compiled and stored on the mobile computing device. The personal medical record is the compilation of multiple medical records including medical data, health-wellness data and dietary data from different sources. The personal medical record may contain information about the social determinants of health about the patient including sex, age, ethnic background, income, work hours, zip code, and the like. Medical data refers to data collected and generated by a care provider. Health-wellness data refers to the “non-medical” data that are generated and collected by individual patients or others (such as family member measuring the vital of the patient). Dietary data refers to the nutritional intake of the patient. For example, medical data from healthcare providers (physician, hospital, etc.), health-wellness data (including fitness data) from different medical devices (e.g. blood pressure monitor, pedometer, etc.), or data from cloud-based storage (such as GOOGLE FIT®), can be compiled in a single personal medical record. Advantageously, the patient maintains custody of the personal medical record. Medical data can include physical activity data (e.g. steps taken, calories burned, distance walked, etc.) and nutritional data (e.g. calories consumed, specific foods consumed, etc.).

In step 104 of method 100, a first survey is constructed that contains questions to allow inference and give explanatory power to the motivation, intention, attitude and ownership of the patient. As used in this specification, these four terms are referred to as the disposition of the patient.

As used in this specification, motivation refers to the patient's interest to self-manage the medical condition. As used in this specification, intention refers to the patient's desire or plan to keep the medical condition under control by adhering to the medical therapy plan. As used in this specification, attitude refers to the patient's perception on positivity and negativity of self-health management. As used in this specification, ownership refers to the patient's belief in accepting responsibility on taking the control on one's health.

In one embodiment, the first survey is given to the patient who supplies personalized answers. In another embodiment, the first survey is sent to many patients for subsequent use in obtaining statistical information. In one embodiment, the survey comprises at least ten questions to permit a meaningful query of multiple dispositions parameters. In step 106, each question in the first survey is assigned four weights: a motivation weight, an intention weight, an attitude weight, and an ownership weight. These weights are a numeric value that scores how heavily the response to a given question should count toward each of these four categories. The weights may be zero weights provided at least one weight is a non-zero weight. In step 108, the answers to the first survey are received by the mobile computing device. In one embodiment, the questions in the first survey are multiple choice questions with a predetermined list of possible answers. This streamlines analysis of the resulting answers. Table 1 provides one illustrative example:

TABLE 1

| Question | |
|---|---|
| number | Question |
| 1 | Have you ever set goal(s) for improving your diet? |
| | Were you successful? Do you intend to start/continue |
| | in the future? |
| 2 | Have you tracked your caloric intake? |
| 3 | Have you ever set goal(s) for improving your exercise/ |
| | fitness level? Were you successful? Do you intend |
| | to start/continue? |
| 4 | Do you know how to track your exercise (e.g. with pen and |
| | paper, or with an electronic device)? If so, how often |
| | do you do it? |
| 5 | Have you exercised at home with(out) the aid of any |
| | instruction (e.g. recorded videos or directions from |
| | a trainer)? |
| 6 | I should take ownership of the data in my health record. |
| 7 | I am responsible for the security and privacy of the health |
| | data under my possession. |
| 8 | I am confident that I can take control to secure and to |
| | protect the privacy of my health data. |
| 9 | How important is diet to you for managing your health? |
| 10 | How important is exercise for managing your health? |
| 11 | How important is monitoring vital signs for managing your |
| | health? |

Table 2 provides examples of a weighting system:

TABLE 2

| Question | | | | |
|---|---|---|---|---|
| number | Motivation | Intention | Attitude | Ownership |
| 12 | 0.44 | 0.35 | 0 | 0 |
| 13 | 0.92 | 0 | 0 | 0 |
| 14 | 0.21 | 0 | 0 | 0 |
| 15 | 0.82 | 0 | 0 | 0 |
| 16 | 0 | 0.35 | 0 | 0 |
| 17 | 0 | 0.45 | 0 | 0 |
| 18 | 0 | 0.75 | 0 | 0 |
| 19 | 0 | 0 | 0.72 | 0 |
| 20 | 0 | 0 | 0 | 0.87 |
| 21 | 0 | 0 | 0 | 0.72 |
| 22 | 0 | 0 | 0 | 0.22 |

In step 110, a baseline motivation score, a baseline intention score, a baseline attitude score and a baseline ownership score is quantified using a combination of the answers to the first survey and the assigned weights. These baseline scores may be personalized for the patient (e.g. the patient answers the first survey) or statistically derived (e.g. multiple patients answer the first survey).

In step 112, the mobile computing device is wirelessly connected to a medical device that generates medical data about the patient. This medical data is stored in the personal medical record. Examples of medical devices include glucose meters, continuous glucose meters, thermometers, pulse oximetry meters, weight scales, blood pressure meters, etc. In one embodiment, a Secure Information Processing with Privacy Assurance (SIPPA) biometric protocol (e.g. a voice biometric protocol) is utilized to encrypt and decrypt the personal medical record. See U.S. Pat. No. 9,197,637. SIPPA services are delivered via a platform solution to a mobile device.

In step 114, medical data is accumulated for at least four days from the medical device(s). Additionally, medical data can be manually entered by the patient. Examples of manually entered medical data can include nutrition information (e.g. food consumed, calories consumed) and similar medical data. A period of at least four days is provided so the patient's behavior is given an opportunity to normalize. Often a patient's behavior changes over time as the patient experiences the medical therapy plan. Step 116 constructs a second survey that is designed to detect these changes.

In step 116, a second survey is constructed and distributed to the patient using the mobile computing device. In one embodiment, the survey comprises at least ten questions to permit a meaningful query of multiple dispositions parameters. The second survey is constructed that contains questions to measure the motivation, intention, attitude and ownership of the patient. The first survey may contain the same questions at the first survey or may contain different questions.

In step 118, each question in the second survey is assigned four weights: a motivation weight, an intention weight, an attitude weight, and an ownership weight. Like the weights assigned in step 106, these weights are numeric values that scores how heavily a response to a given question should count toward each of these four categories.

In step 120, the answers to the second survey is received by the mobile computing device. The user may enter these answers using a user interface (e.g. keyboard, touch screen, etc.) that is part of the mobile computing device.

In step 122, updated scores are calculated based on the answers to the second survey and the corresponding weights. The updated scores are an updated motivation score, an updated intention score, an updated attitude score and an updated ownership score. These baseline scores are personalized for the patient because they are based on the answers to the second survey that were provided by the patient.

In step 124, the baseline scores and the updated scores in each of the four categories are compared to quantify score changes. This comparison permits quantification of the patient's change in motivation, change in intention, change in attitude and change in ownership.

In step 126, a message is displayed to the patient on the mobile computing device. The message is customized based on the second set of answers and the change in motivation, change in intention, change in attitude and change in ownership. In one embodiment, the answer is also customized based on medical data that was accumulated during the at least four days. These customized messages may be displayed during key pauses in transition to other functions of the mobile application. For example, the mobile application may pause while survey answers are uploaded to a remote server for processing or when data is exchanged with the medical device in step 112 or step 114. Additionally or alternatively, the mobile application may pause during encryption or decryption of the personal medical record. The customized message may be displayed during these, or other, pauses. In one embodiment, the pause is intentionally extended to provide at least five seconds for the patient to read the customized message.

The mobile computing device allows the patient to access multiple personalized services ranging from medication research, reminder services, import/exchange health data in an interoperable format under common standard of Meaningful Use. Examples of medication research include loading a webpage with information pertaining to a medication that is taken as part of the medical therapy plan. Examples of reminder services including triggering an alarm on the mobile computing device on a predetermined schedule to remind the patient to take a medication or engage in a particular physical exercise. Examples of exchanging health data include connecting, by a wired or wireless connection, to an external computer at a medical care provider's location.

The disclosed platform enables a patient centric approach for privacy preserved data collection to gain understanding on the impact of social, economic, and “non-clinical” behavioral lifestyle considerations on health. In one embodiment, the platform utilizes Structure Equation Modeling (SEM) to analyze the survey results and apply appropriate weights. The origin of SEM is evolved out of research across various disciplines. This research follows the Linear Structural Relations (LISREL) model for SEM that takes into consideration of measurement errors in observed variables, but could be simplified if measurement error is negligible.

In general, SEM consists of two parts. The first part is a set of equations that give the causal relations between the substantive variables of interest, referred to as “latent variables,” which are not observable. In the disclosed case, this includes attitude, intention, motivation, and ownership (regarding taking control). The latent variable model gives the causal relationships between these variables when the measurement error is absent or negligible. Mathematically, it is represented as below:

ηᵢ=αₙ+Bηᵢ+Γξᵢ+γᵢ (1)

In the equation above, ηᵢ, is the i^{th} vector of latent (endogenous) variables. αₙ is the vector of intercepts. B is the matrix of coefficients that give the expected effect of the ηᵢ on ηᵢ where its main diagonal is zero. is the vector of latent exogenous variables. Γ is the matrix of coefficients that give the expected effects of ξᵢ on ηᵢ. γᵢ is the vector of equation characterizing the disturbances that consists of all other influences on ηᵢ not included in the equation. Furthermore, the latent variable model also assumes that the mean of the disturbances is zero (i.e., E(γᵢ)=0) and that the disturbances are uncorrelated with the latent exogenous variables (i.e., Cov(γᵢ, ξᵢ)=0). If the Coy is not zero, then those variables are correlated with the disturbances are not exogenous and are included as an endogenous latent variable in the model. This is the case in regard to the latent variable ownership.

While the elements of the covariance matrices of ξᵢ and γᵢ could be manually determined to be freely estimated, constrained to zero or other values, one embodiment of the disclosed system uses LISREL software to make the determination.

The second part of SEM connects the observed variables with the latent variables as below:

yᵢ=α_{y}+Λ_{y}ηᵢ+εᵢ (2)

xᵢ=αₓ+Λₓξᵢ+δᵢ (3)

In the measurement model above, xᵢ and yᵢ are the vectors of indicators of ξᵢ and ηᵢ respectively. αₓ and α_{y} are the vectors of intercepts. Λ_{y} is the loading factor matrix that gives the expected effects of ηᵢ on yᵢεᵢ is the vector of disturbances consisting of influences on yᵢ that are not part of ηᵢ. Λₓ is the loading factor matrix that gives the expected effects of ξᵢ on xᵢ. δᵢ is the vector of disturbances consisting of all influences on xᵢ that are not part of ξᵢ. Finally, the measurement model assumes a zero mean of disturbances and different disturbances are uncorrelated. Again, the elements of the covariance matrices of εᵢ and δᵢ could be manually determined to be freely estimated, constrained to zero or other values.

In SEM, one could incorporate causal assumptions as part of the model. This is the disclosed case. A causal relationship is assumed between motivation, intention, ownership and attitude. The measurement errors of all variables is assumed to be negligible or zero. This is achieved through manual filtering of obvious pilot data that are error prone; e.g., contradictory responses. This allows one to set yᵢ=ηᵢ and xᵢ=ξᵢ and reduce the SEM formulation to below:

yᵢ=αₙ+ByᵢΓᵢxᵢ+γᵢ (4)

In the practice of SEM approach, one can choose to make strong or weak causal assumptions, as well as whether two disturbances are uncorrelated or not. This disclosure utilizes assumptions that yielded the “best” model in terms of statistical power and goodness of fit.

In brief, each (non-observable) behavior construct (motivation, intention, attitude and ownership) corresponds to one or more survey questions (MOT_xx, INT_xx, ATT_xx). Each possible response to a survey question is designed and is gone through a team discussion on its relevancy to the behavior constructs: motivation, intention, attitude and ownership and assigned the aforementioned weights. Further details on this model will be given in the next section.

Pilot Study and Sippa-Sem-TPB Model Validation

The development of the Structure Equation Model discussed in this disclosure is based on the data collected under an Institutional Review Board (IRB) sanctioned pilot. This pilot consisted of five components below:

Component 1: Initial screen survey that consisted of 30 questions for polling data related to eligibility, chronic conditions, social determinants and lifestyle.

Component 2: Orientation for enrolled participants, installation and configuration of a mobile application, as well as the collection of informed consent.

Component 3: Pre-pilot 13-question survey with questions related to motivation, intention, attitude and ownership.

Component 4: Remote self-guided exploratory session to carry out five specific tasks using the mobile application, as well as participating in an exit survey.

Component 5: Post-pilot de-brief interview.

@Component 1: Approximately 500 subjects participated in an initial online screen survey. Their responses form the basis for the development of the disclosed SIPPA-SEM-TPB model. These subjects were recruited from multiple sites. 38% of them were female. About 50% had a household income of less than $50K, 30% between $50K and $100K, and 20% had a household income of over $100K. About 44% of the population had less than 2 years of college study, 36% had two to four years of college study, and 20% had been in a graduate program. 15% reported to work/study over 50 hours a week, 35% between 36 hours and 50 hours, and 50% of them work/study less than 36 hours a week.

Among the 500 subjects, approximately 120 subjects completed only partially the screen survey. Among the rest, 84 subjects expressed interest and satisfied the inclusion/exclusion criteria to be enrolled. Because the pilot requires participants to engage with an ANDROID® application, the inclusion criteria included (1) age 18 or older, (2) basic internet computing skill and (3) the possession of an ANDROID® device.

@Component 2: Each of the 84 subjects enrolled into the pilot was asked to sign the informed consent, and was assigned to one of four handlers in this research team.

The handlers contacted subjects by email, and arranged a schedule for an orientation. During the orientation, a subject returns the signed informed consent, and works with the handler to install and configure the mobile application, as well as to download two test patient health records. The subject is also notified that the mobile application will track the meta-data of the usage such as the time and date, as well as the usage frequency of each service of the application. No sensitive or private information will be recorded.

A handler also gave a demonstration of the mobile application and walked through the steps for using the mobile application on the following five tasks. (1) Import, encrypt, decrypt, and view a test health record in interoperable format CCD. (2) Consolidate the information in the second test health record by merging two records. (3) Research medication information of interest, and set reminders for medication adherence. (4) Participate in online survey delivered to the mobile application about nutrition education and therapy. (5) Participate in a video conference to simulate the interaction between a patient and a remote healthcare provider through teleconsultation.

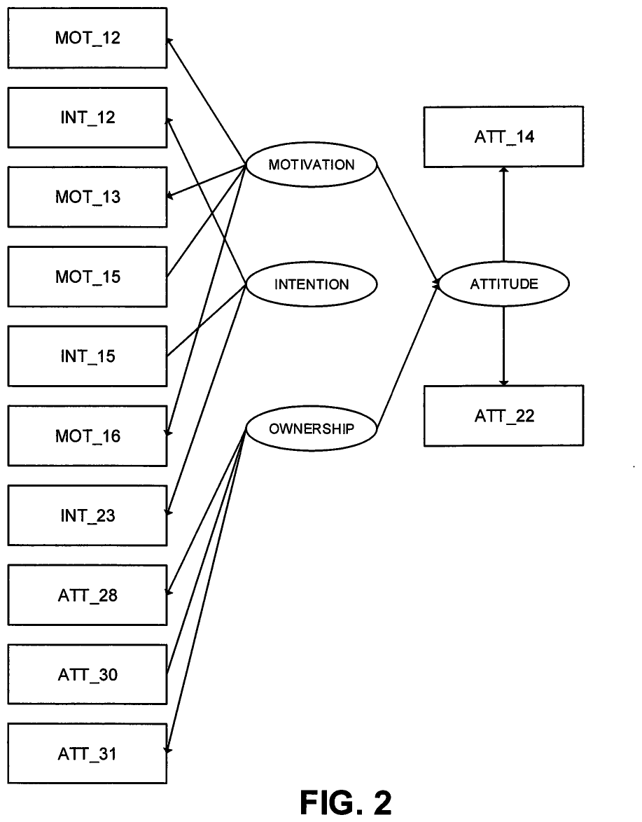

@Component 3: A pre-study survey of 13 questions extracted from the screen survey was provided to each subject. The survey response from each subject was used to establish a baseline about the level of engagement quantified in terms of the behavior constructs modeled as latent variables in FIG. 2. Specifically, an inverse SIPPA-SEM-TPB model was derived to predict quantifiable behavior constructs; i.e., given a survey response and the inverse model, linear regression could be performed to derive the quantified behavior constructs.

The basis for deriving the inverse model is the SIPPA-SEM-TPB model developed using the response data of approximately 500 participants in component 1. The architecture of one version of the model is shown in FIG. 2. For example, questions 12, 13 and 16 were deemed relevant to motivation (MOT_12, MOT_13 and MOT_16). Questions 12, 15 and 23 were deemed relevant to intention (INT_12, INT_15 and INT_23). Note question 12 was relevant to both motivation and intention. Questions 28, 30 and 31 were deemed relevant to ownership (ATT_28, ATT_30, ATT_31). Questions 14 and 22 were deemed relevant to attitude (ATT_14 and ATT_22). Both ownership and motivation were also factors in determining the patient's attitude score.

The model is validated following the criteria and thresholds commonly agreed upon in the research community:

| Criteria for good fit/significance | |
|---|---|
| At degree of freedom = 42, | SIPPA-SEM-TPM |
| 1. Alpha = 0.0025 ⇔ Chi-square = 72.32 | Chi-square = 101.32 |
| Alpha = 0.005 ⇔ Chi-square = 69.336 | |
| p-value < 0.005 | p-value = 0 |
| RMSEA < 0.06 | RMSEA = 0.054 |

@Component 4: Each subject was asked to conduct a self-guided exploratory session. In this study, the self-guided exploratory session is no less than 4 days but no more than 17 days unless there is a special circumstance. During the self-guided exploratory session, a subject would interact with up to three surveys delivered online directly to their mobile computing device through a SIPPA Health server. These surveys targeted nutrition education and therapy, and engaged subjects to set goals for increasing whole grain intake. After the self-guided exploratory session is completed, each subject was asked to complete a post-study survey that is identical to the pre-study survey. Among the 84 subjects, only the data from 52 subjects was actually used in this pilot study. The data from the rest of the subjects was not usable due to various reasons ranging from missing survey responses to contradictory responses; e.g., one responded “I know how to track my caloric intake and I do it almost every day.” and “I've never tracked my caloric intake.” in the pre-study and post-study survey, respectively.

Of the 52 subjects, the quantitative measures on the motivation, intention and attitude are again derived from the inverse model for each individual, and compared against the individual's baseline obtained from the pre-study response. The quantitative changes on motivation (ΔMot), intention (ΔInt), attitude (ΔAtt), and ownership (ΔOwn) are computed, resulting in 52 data points on the change for each behavior construct. The correlations among the behavior constructs were investigated using (1) all 52 data points, (2) only the data points from those who self-reported to have at least one chronic condition, and (3) only the data points from those who self-reported to have no chronic condition. The results are tabulated and shown below:

TABLE 3
Correlation using all data

| | | | | |
|---|---|---|---|---|
| Corr(ΔMot, ΔAtt) | 0.58 | | | |
| Corr(ΔMot, ΔInt) | | 0.30 | | |
| Corr(ΔInt, ΔAtt) | | | 0.19 | |
| Corr(ΔMot, ΔOwn) | | | | 0.24 |

TABLE 4
Correlation using data of those with chronic conditions

| | | | | |
|---|---|---|---|---|
| Corr(ΔMot, ΔAtt) | 0.30 | | | |
| Corr(ΔMot, ΔAtt) | | 0.43 | | |
| Corr(ΔInt, ΔAtt) | | | 0.07 | |
| Corr(ΔMot, ΔOwn) | | | | −0.03 |

TABLE 5
Correlation using data of those without chronic condition

| | | | | |
|---|---|---|---|---|
| Corr(ΔMot, ΔAtt) | 0.67 | | | |
| Corr(ΔMot, ΔInt) | | 0.29 | | |
| Corr(ΔInt, ΔAtt) | | | 0.24 | |
| Corr(ΔMot, ΔOwn) | | | | 0.31 |

@Component 5: At the end of the study, each participant is scheduled for a de-brief interview to gather information that could not be captured statistically, and that could be used to check the consistency of the quantitative data captured.

The utility of the validated SIPPA-SEM-TPB model is illustrated through a novel application of software development. More specifically, this disclosure illustrates the incorporation of behavior constructs into the consideration of strategy design pattern of Unified Modeling Language (UML) for developing Digital-Health-Software-as-a-Service (DHSaS).

According to an article in HealthIT news in 2015, there were 165,000 health-related mobile applications available. About a quarter of the mobile applications are related to chronic disease management. Yet only 0.022% of the mobile applications—36 out of 165,000—account for 50% of all those downloaded. While most mobile applications arguably attempt to facilitate information communication, few may have incorporated a design that makes explicit the objective of affecting healthcare outcome. There are even fewer mobile applications that achieve usability as measured by the retention rate. Currently a mobile application that can achieve a retention rate of 25% is considered a big success; i.e., 25% of the users who download a mobile application use it on a daily basis. The disparity between the number of mobile applications available and the number of mobile applications being used actively could be attributed to: (1) Lack of motivation for an individual to engage in healthy behaviors. (2) Disconnection between the perceived value of digital health and an individual; thus lack of intention to acquire the behavior health skill needed to engage in a health intervention.

To alleviate these problems, SIPPA-SEM-TPB could be applied to discover the motivation indicator of an individual to improve patient engagement, as well as to incorporate the characteristics of behavior constructs, that aligns with the motivation indicator, into the software specification in the development process of the digital health software services. One such use case is illustrated below.

As described in the previous section, the inverse model of SIPPA-SEM-TPB was applied to identify change in motivation, intention and attitude. Data analytics was applied to discover the statistically significant association patterns that could be used to inform software specifications formulated in terms of strategy design pattern in UML. The concept of association pattern discovery can be described via an example below:

Assume a survey similar to the one described in the previous section was conducted. The response to the survey questions by a patient could be represented as (X1:val^{X1}ᵢX2: val^{X2}ⱼ . . . Xn: val^{Xn}ₖ); where X1 . . . Xn are the variables corresponding to the survey questions. A collection of responses to the survey becomes a data set on (X1 X2 . . . Xn). A statistical association measure for (X1:val^{X1}ᵢ . . . Xp: val^{Xp}ₖ) is considered α-significant if the following two conditions are satisfied:

1. The support for (X1:val^{X1}ᵢ . . . Xp: val^{Xp}ₖ), defined as Pr(X1:val^{X1}ᵢ . . . Xp: val^{Xp}ₖ), is at least α; i.e., Pr(X1:val^{X1}ᵢ . . . Xp: val^{Xp}ₖ)≥α.

2. The interdependency of {X1:val^{X1}ᵢ . . . Xp: val^{Xp}ₖ} as measured by mutual information measure MI(X1:val^{X1}ᵢ . . . Xp: val^{Xp}ₖ)=Log₂ Pr(X1:val^{X1}ᵢ . . . Xp:val^{Xp}ₖ)/Pr (X1:val^{X1}ᵢ) . . . Pr(Xp: val^{Xp}ₖ)≥β(χ²)^{γ}; where β and γ are some scaling factors, and due to Pearson, χ²=(oi−ei)²/ei.

The technical details for discovering association patterns can be referred to the textbook Bon K. Sy, Arjun Gupta, Information-Statistical Data Mining and Oracle Basics for Warehouse Building, Springer ISBN: 978-1-4613-4755-2 (Print) 978-1-4419-9001-3 (Online), 2003/2004.

Association pattern discovery was applied to data collected from the IRB sanctioned pilot study. Three exemplary patterns are shown below that were selected from a set of 12 (and 10) statistically significant association patterns discovered out of 160 possible second order association patterns for the chronic (non-chronic) population:

For the population with at least one chronic condition(s):

TABLE 6
Association patterns of population with chronic disease

| | | | | Chi- | | assoc − |
|---|---|---|---|---|---|---|
| ΔInt | ΔOwn | pr(ΔInt, ΔInt) | Assoc | square χ² | χ²/2N | χ²/2N |
| 2 | 3 | 0.1818 | 0.5525 | 0.2970 | 0.0133 | 0.539 |
| 3 | 1 | 0.1818 | 0.8745 | 0.7576 | 0.0345 | 0.840 |

For the population without chronic condition:

TABLE 7
Association patterns of population without chronic disease

| | | | | Chi- | | assoc − |
|---|---|---|---|---|---|---|
| ΔInt | ΔOwn | pr(ΔInt, ΔInt) | Assoc | square χ² | χ²/2N | χ²/2N |
| 2 | 2 | 0.2195 | 0.027 | 0.3164 | 0.0038 | 0.2662 |

Because change in intention (ΔInt) is associated with change in ownership to take control (ΔOwn), and by cross referencing and comparing the correlation derived from the population data between the chronic and non-chronic patient population, one of the interesting findings is reviewed by the following analysis: Individuals with a chronic condition shows a stronger ownership on achieving adherence as evidence by a strong correlation between the usage of medication reminder and the change in motivation (0.2035 vs 0.0323).

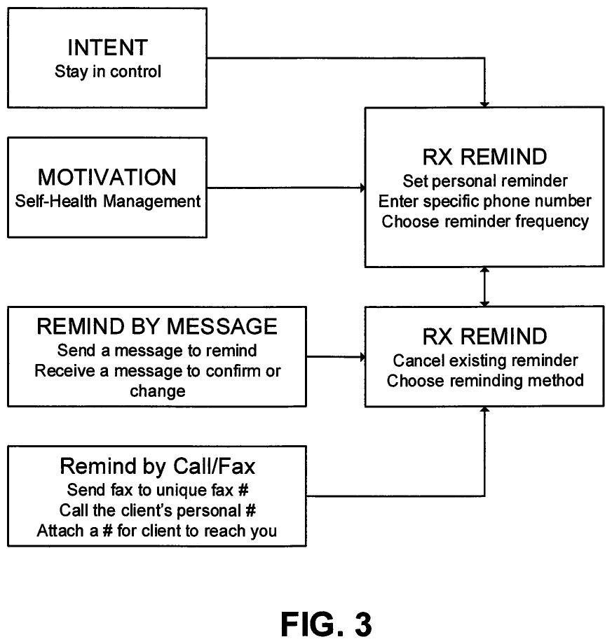

Once the finding is confirmed that there is an alignment between the motivation indicator of the individuals with chronic conditions and the reminder (service), strategic design pattern of UML (Unified Modeling Language) in software engineering is applied to develop the reminder digital health service. One such design is shown in FIG. 3. The illustrated design considers motivation, intention, attitude and ownership to determine a fit between a given feature or software service and the patient's interaction activity level. It is possible that a software service is essential for a patient to maintain health, while the utilization of the service is low. For example, some patients may utilize the mobile application for two days and then rarely use it again. In such a case, a decrease in intention and motivation is detected. This causes the mobile application to identify alternative software feature(s) that offer a functionality that is equally attractive while in alignment with the discovered motivation indicators. The mobile application will send customized messages to the patient to consider the alternative software feature and offers assistance to re-customize the mobile application. If there is no alternative software feature while the utilization is important to the patient's health, the mobile application will send customized messages to both the patient and the significant others to encourage additional activity (e.g. encourage updating medical data, etc.). In contrast, different patients may utilize the mobile application frequently. This causes the mobile application to maintain its current customized messages because those customized messages have been found to promote frequent use.

The following illustrative examples are provided to depict how the disclosed method can function.

### Example 1

A patient launches the mobile application on a tablet device. The patient is engaging in a medical therapy plan that involves changes in diet and exercise to control high blood pressure. In addition, a prescription drug is being given. The mobile application decrypts a personal medical record on the tablet. The mobile application then wirelessly connects to a medical device, such as a blood pressure monitor, that is connected to the patient. The blood pressure monitor measures the patient's blood pressure and wirelessly updates the personal medical record with the blood pressure measurement. The mobile application then encrypts the personal medical record.

### Example 2

The patient of Example 1 continues to collect blood pressure measurements over a period of two months. During a doctor's visit, the patient electronically sends to a computer at the doctor's office and updates both the personal medical record, and the doctor's electronic medical record, with updated medical data. This permits the doctor access to detailed medical records with the self-monitoring data carried out at home during the period of two months.

### Example 3

The patient of Example 2 continues to use the mobile application for an additional three months. During this time the patient completes two surveys, each of which are compared to a previous survey that established a baseline disposition of the patient. Based on this comparison a negative change in motivation was detected. This was accompanied by a modest increase in blood pressure, as determined by blood pressure measurements. Based on this loss of motivation, a customized message is generated that is displayed during the next exchange of medical data with the blood pressure monitor. The message reminds the patient that the mobile application can provide reminders to measure blood pressure or to take the prescription drug using text messages, automated voice calls or smartphone alarms. Because the use of the mobile application's reminder system is known to be positively corrected with motivation, this may counteract the negative change in motivation. Based on the elevation in blood pressure, the message may also remind the patient that certain nutritional habits (e.g. reduce sodium intake) can help one control high blood pressure.

### Example 4

In one embodiment, the message is customized based on the answers to the most recent survey and the change in disposition. The patient of Example 3 answers “I am not willing” to the question of “How willing are you to modify your eating habits?” Based on the patient's unwillingness as detected in the most recent survey, the customized message is further customized to only remind the patient that the mobile application can provide reminders to measure blood pressure or to take the prescription drug. The customized message omits reminders that certain nutritional habits (e.g. reduce sodium intake) can help one control high blood pressure because this reminder will prove to be unfruitful.

### Example 5

The patient of Example 4 configures the mobile application to provide reminders in accordance with the medical therapy plan. The mobile application may be configured to send reminders to take the prescription drug in a timely manner. The reminder may be an automated voice of facsimile telephone call, a timed alarm, a text message or similar reminder. The mobile application may also be configured to send reminders to record the patient's blood pressure using the blood pressure monitor.

### Example 6

The patient of Example 5 visits a second doctor. Because the personal medical record is an aggregation of data from multiple sources, the personal medical record provides the patient with the ability to compile a complete medical record while safely and securely maintaining total control over the record itself on their local mobile computing device. This provides a significant advantage over services that store the patient's medical information on a remote server. In such remote systems the patient does not have personal custody of the medical records and is at the mercy of the host of the remote server.

### Example 7

The patient of Example 7 is from a Chinese culture and this information is stored in the personal medical record. Culturally it is very difficult to ask Indian or Chinese patient to not eat rice. Asking dietary control by removing rice as part of a meal will have a probability of failure. Accordingly, a customized message that suggests certain meals would disregard or minimize sending messages that promote rice-free meals.

### Example 8

The patient of Example 8 works 10 hours per day, 7 days a week and this information is stored in the personal medical record of health and social determinants of health. The mobile application determines the patient is, therefore, unlikely to find time to exercise. If the mobile application proposes exercise without offering time management advice, the medical therapy plan is bound to fail. Accordingly, the mobile application sends a customized message to the patient to probe the patient's daily activity and propose alternatives to exercise, such as healthy eating.

### Example 8

The patient of Example 9 is the primary income earner in the household and this information is stored in the personal medical record of the patient. The personal medical record includes certain social determinants of health, specifically the patient's annual income. This patient earns $40,000 per year. The mobile application determines the patient is unlikely to spend $400 on a monthly supply of a single medication. Accordingly, the mobile application sends a customized message that encourages non-pharmacological treatment, encourages exercise and healthy eating.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a non-transient computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language, mobile application development such as ANDROID® programming language, front end programming language such as Angular or React Native, or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A mobile computing device configured to execute a method for enhancing patient compliance with a medical therapy plan, the method comprising steps of:
compiling a personal medical record that is stored on the mobile computing device owned by the patient, the personal medical record comprising at least one of medical data, health-wellness data or dietary data of the patient;
constructing a first survey comprising a first plurality of questions;
receiving a first set of answers concerning the first survey;
wirelessly connecting the mobile computing device to at least one digital medical device;
accumulating medical data over a period of at least four days from the at least one medical device and updating the personal medical record with the medical data, the health-wellness data or the dietary data;
constructing a second survey comprising a second plurality of questions;
receiving a second set of answers from the patient concerning the second survey, wherein the second survey is spaced at least four days after the first survey;
quantifying, using a Structure Equation Model (SEM), a change in a disposition of the patient by comparing the first set of answers to the second set of answers;
displaying a message to the patient on the mobile computing device wherein the message is customized based on the second set of answers and the change in disposition.

2. The mobile computing device of claim 1, wherein the first plurality of questions in the first survey include multiple choice answers.

3. The mobile computing device of claim 1, wherein the step of accumulating medical data accumulates medical data from at least two different medical devices over the period of at least four days.

4. The mobile computing device of claim 1, wherein the step of accumulating medical data accumulates medical data from at least a pedometer.

5. The mobile computing device of claim 1, wherein each of the first survey and the second survey comprise at least ten questions.

6. The mobile computing device of claim 1, further comprising a step of encrypting the personal medical record.

7. The mobile computing device of claim 6, wherein the step of encrypting uses biometric encryption.

8. The mobile computing device of claim 6, wherein the step of encrypting uses biometric encryption including at least one of a voice modality, a fingerprint modality, or a face modality.

9. The mobile computing device of claim 6, wherein the step of display the message occurs during the step of encrypting.

10. The mobile computing device of claim 1, further comprising a step of pausing operation of the mobile computing device, wherein the step of display the message occurs during the step of pausing.

11. The mobile computing device of claim 10, wherein the step of pausing pauses for at least five seconds.

12. The mobile computing device of claim 1, wherein the method further comprises a step of manually entering supplemental medical data into the personal medical record, wherein the step of manually entering is performed by the patient.

13. The mobile computing device of claim 11, wherein the message is customized based on the second set of answers, the change in disposition and the supplemental medical data.

14. The mobile computing device of claim 1, wherein the mobile computing device is configured to provide video chat with a healthcare provider.

15. The mobile computing device of claim 1, wherein the personal medical record comprises social determinants of health.

16. The mobile computing device of claim 15, wherein the social determinants of health comprises sex and age.

17. The mobile computing device of claim 16, wherein the social determinants of health further comprises ethnic background.

18. The mobile computing device of claim 16, wherein the social determinants of health further comprises at least one socioeconomic factor selected from annual income, weekly work hours and zip code.

19. A mobile computing device configured to execute a method for enhancing patient compliance with a medical therapy plan, the method comprising steps of:
compiling a personal medical record that is stored on the mobile computing device owned by the patient, the personal medical record comprising medical data, health-wellness data or dietary data of the patient;
constructing a first survey comprising a first plurality of questions;
assigning each question in the first plurality of questions a first motivation weight, a first intention weight, a first attitude weight and a first ownership weight;
receiving a first set of answers concerning the first survey;
quantifying, using a Structure Equation Model (SEM), (1) a baseline motivation score using the first set of answers and the first motivation weight (2) a baseline intention score using the first set of answers and the first intention weight (3) a baseline attitude score using the first set of answers and the first attitude weight (4) a baseline ownership score using the first set of answers and the first ownership weight;
wirelessly connecting the mobile computing device to at least one digital medical device;
accumulating medical data over a period of at least four days from the at least one medical device and updating the personal medical record with the medical data, the health-wellness data or the dietary data;
constructing a second survey comprising a second plurality of questions;
assigning each question in the second plurality of questions a second motivation weight, a second intention weight, a second attitude weight and a second ownership weight;
receiving a second set of answers from the patient concerning the second survey, wherein the second survey is spaced at least four days after the first survey;
quantifying (1) an updated motivation score using the second set of answers and the second motivation weight (2) an updated intention score using the second set of answers and the second intention weight (3) an updated attitude score using the second set of answers and the second attitude weight (4) an updated ownership score using the second set of answers and the second ownership weight;
quantifying a change in the motivation, a change in the intention, a change in the attitude and a change in the ownership based on a comparison of (1) the baseline motivation score and the updated motivation score, (2) the baseline intention score and the updated intention score, (3) the baseline attitude score and the updated attitude score, and (4) the baseline ownership score and the updated ownership score, respectively;
displaying a message to the patient on the mobile computing device wherein the message is customized based on the second set of answers and the change in the motivation, the change in the intention, the change in the attitude and the change in the ownership.

20. The mobile computing device as recited in claim 19, wherein the Structure Equation Model (SEM) comprises latent variables and observed variables.
